(19) United States
(12) Patent Application Publication
(10) Pub. No.: US 2006/0235430 A1
(43) Pub. Date: Oct. 19, 2006

(54) Corneal implant injector assembly and methods of use

(75) Inventors: Alan Ngoc Le, Lake Forest, CA (US); Jeff Forsberg, Huntington Beach, CA (US); Crystal M. Cunanan, Mission Viejo, CA (US); Alexander Vatz, Lake Forest, CA (US)

Correspondence Address: ORRICK, HERRINGTON & SUTCLIFFE, LLP;IP PROSECUTION DEPARTMENT, 4 PARK PLAZA, SUITE 1600, IRVINE, CA, 92614-2558, US

(73) Assignee: INTRALENS VISION, INC.

(21) Appl. No.: 11/107,069

(22) Filed: Apr. 15, 2005

(51) Int. Cl. A61F 9/00

(52) U.S. Cl. 606/107

(57) ABSTRACT

A corneal implant injector assembly includes a barrel, a plunger, and an injector tip with a channel having a size and orientation adapted to store and deliver a corneal implant. The corneal implant is preferably stored in the channel in a contracted state. The plunger has an implant engagement tip for engaging and moving the implant within the channel and to deploy the implant. In some embodiments, the injector tip is selectively detachable from the remaining portion of the injector assembly.

### FIELD OF THE INVENTION

The present invention relates to apparatus, systems, and methods for implanting corneal implants to the corneal surface of the eye.

### BACKGROUND OF THE INVENTION

The eye works on a principle very similar to that of a camera. The iris—the colored portion of the eye about the pupil—functions like a shutter to regulate the amount of light admitted to the interior of the eye. The cornea and natural lens focus the rays of light on the retina. The retina then transmits the image of the object viewed to the brain via the optic nerve. Normally, these light rays are focused exactly on the retina, which permits the distant object to be seen distinctly and clearly. Deviations from the normal shape of the corneal surface, however, produce errors of refraction in the visual process so that the eye becomes unable to focus the image of the distant object on the retina. Hyperopia, or “farsightedness,” is an error of refraction in which the light rays from a distant object are brought to focus at a point behind the retina. Myopia, or “nearsightedness,” is an error of refraction in which the light rays from a distant object are brought to focus in front of the retina, such that when the rays reach the retina they become divergent, forming a circle of diffusion and, consequently, a blurred image.

In recent years, as refractive surgery has developed, a number of surgical techniques have become available to surgically treat nearsightedness, farsightedness, and astigmatism. For example, corneal implants are used to correct visual disorders such as myopia, hyperopia, presbyopia (difficulty in accommodating a change in focus), and astigmatism. To correct these disorders, an implant is introduced into the body of the cornea in known ways, such as after a flap is formed in the cornea and the cornea is exposed. The implant changes the shape of the cornea and alters its refractive power. These implants are generally made of hydrogels but can include other polymers, tissue implants, or the like.

Corneal implants have typically been stored free-floating in a volume of storage fluid contained within a storage container. To retrieve the implant, one had to first locate the implant within the fluid, then remove the implant using a filter device or sequestering tool. In the case of a corneal implant, locating the implant is complicated by both the size and the transparency of the implant. For example, a corneal implant generally has a diameter of about 4.0 to about 7.0 mm and a center that is normally fabricated having a thickness ranging from about 25 to about 50 microns. Due to this small size, physically grasping the implant from the storage fluid using tweezers, or some similar operation, is simply not practical.

Isolation of a corneal implant, or other specimen, has generally required the use of a sieve to separate the implant from the fluid. Isolating the implant in this manner, however, subjects the implant to mechanical forces, which could lead to a loss of the implant. If not damaged, the transparent implant must still be located on the sieve surface and retrieved. The implant must therefore be grasped using tweezers, forceps, or the like. Imparting such force upon the implant, however, can also damage the implant. Using force imparting tools to hold the implant is therefore not desirable. Prior isolation techniques were therefore difficult, time-consuming, and created additional steps, which could also lead to implant contamination. Thus, it has been desired to have an implant storage and handling system that allows the user to rapidly and successfully retrieve the implant for prompt implantation.

Prior devices used to deposit an implant onto the cornea surface have typically placed the corneal implant onto the cornea surface in a bunched or folded conformation. Aligning the implant in planar relation to the cornea surface required the surgeon to manipulate or tease the implant so as to remove any folds or bends in the implant. Problematically, the step of unfolding the implant on the cornea surface could cause serious trauma to the cornea surface. This trauma can lead to the formation of edema, or other deleterious responses that lead to rejection or displacement of the implant.

Thus, a need has existed for a unitary packaging and handling system that provides the desired storage capabilities, easy retrieval of the specimen from the storage, and tools that are operable to retrieve and utilize the specimen without causing damage to the specimen or an implantation site. In addition, a need has existed for a more effective method for implanting or depositing a corneal implant onto a corneal surface.

In response to these needs, the current Applicant has previously developed a “System for Packaging and Handling an Implant and Method of Use,” as described in U.S. patent applicant Ser. No. 10/999,093, filed on Nov. 29, 2004, (“the '093 application”), which application is hereby expressly incorporated by reference herein in its entirety. The foregoing application describes an implant packaging and handling system that includes a storage bottle having an opening to receive a volume of implant storage fluid, and an implant holding tool designed to retain the implant in fluid communication with the implant storage fluid. The implant holding tool includes a retaining member detachably mounted to an implant applicator tool. While the systems and methods described in the '093 application provide solutions to several of the problems with the previous systems and methods, additional improvements are desired.

### SUMMARY OF THE INVENTION

The present invention provides improved apparatus, systems, and methods for storing and retrieving a corneal implant and for depositing an implant onto the cornea during a refractive surgical procedure. The apparatus, systems, and methods provide for improved implant storage and retrieval capabilities over those of the prior art, and provide improved methods for deploying corneal implants during ophthalmologic surgical procedures.

Generally, an apparatus of the present invention is suitable for deploying a corneal implant. The apparatus includes a barrel portion, a plunger at least partially disposed within a portion of the barrel, and an injector tip formed integrally with or detachably attached to a distal portion of the barrel. The injector tip preferably includes a channel having a size and orientation adapted to store and then deploy a corneal implant. The plunger may be either a single structure or a combination structure, and preferably has an engagement tip adapted to safely engage and move the implant within the injector tip channel. The injector tip may advantageously be detachable from the remainder of the injector assembly so that the tip (with the implant already contained in the channel) may be stored separately in a container charged with a storage medium. Alternatively, the entire injector assembly may be stored in the storage medium in cases where the injector tip is or is not detachable from the remainder of the assembly.

A method of the present invention includes forming a bed or channel on or in the cornea of an eye, placing the delivery tip region of the injector assembly in proximity to the bed or channel, deploying the corneal implant to the bed or channel, then adjusting the position of the corneal implant after deployment.

Other systems, methods, features and advantages of the invention will be or will become apparent to those skilled in the art upon examination of the following figures and detailed description of the preferred embodiments. It is intended that all such additional systems, methods, features, and advantages be included within this description, be within the scope of the invention, and be protected by the accompanying claims. It is also intended that the invention not be limited to the details of the example embodiments.

### BRIEF DESCRIPTION OF THE DRAWINGS

FIG. 1A shows perspective view of an embodiment of a lens injector assembly in accordance with the present invention.

FIG. 1B shows a partially exploded view of the lens injector assembly of FIG. 1A.

FIG. 1C shows a cross-sectional view of the lens injector assembly of FIG. 1A.

FIG. 1D shows a side view of a barrel.

FIG. 1E shows a side view of a plunger.

FIG. 1F shows a side view of a tip.

FIG. 1G shows a cross-sectional view of a tip taken at line G-G shown in FIG. 1F.

FIG. 2 shows an exploded view of another embodiment of a lens injector assembly in accordance with the present invention.

FIG. 3 shows a side view of the lens injector assembly of FIG. 2 and a stabilizer and vial.

FIG. 4A shows a vial containing a tip portion of the lens injector assembly of FIG. 2.

FIG. 4B shows the vial of FIG. 4A with the stopper removed and the injector assembly tip withdrawn from the vial.

FIG. 4C shows a side view of the tip portion of the lens injector assembly of FIG. 2.

FIG. 5A shows a side view of another embodiment of a lens injector assembly in accordance with the present invention.

FIG. 5B shows another side view of the lens injector assembly of FIG. 5A.

FIG. 5C shows an exploded view of the lens injector assembly of FIG. 5A.

FIG. 5D shows a close-up view of the tip portion of the lens injector assembly of FIG. 5A.

FIG. 5E shows another close-up view of the tip portion of the lens injector assembly of FIG. 5A after deployment of a lens.

FIG. 6A shows a cross-section view of a corneal implant in its expanded state.

FIG. 6B shows a cross-sectional view of a corneal implant in a compacted state.

FIG. 7 shows a side view of another embodiment of a lens injector assembly including a bellows structure.

FIG. 8 shows a side view of another embodiment of a lens injector assembly including an implant adjustment member.

FIG. 9 shows a cap suitable for use with a lens injector assembly.

### DETAILED DESCRIPTION OF THE PREFERRED EMBODIMENTS

Before the present invention is described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which these inventions belong. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise.

As will be apparent to those of skill in the art upon reading this disclosure, each of the individual embodiments described and illustrated herein has discrete components and features which may be readily separated from or combined with the features of any of the other several embodiments without departing from the scope or spirit of the present inventions.

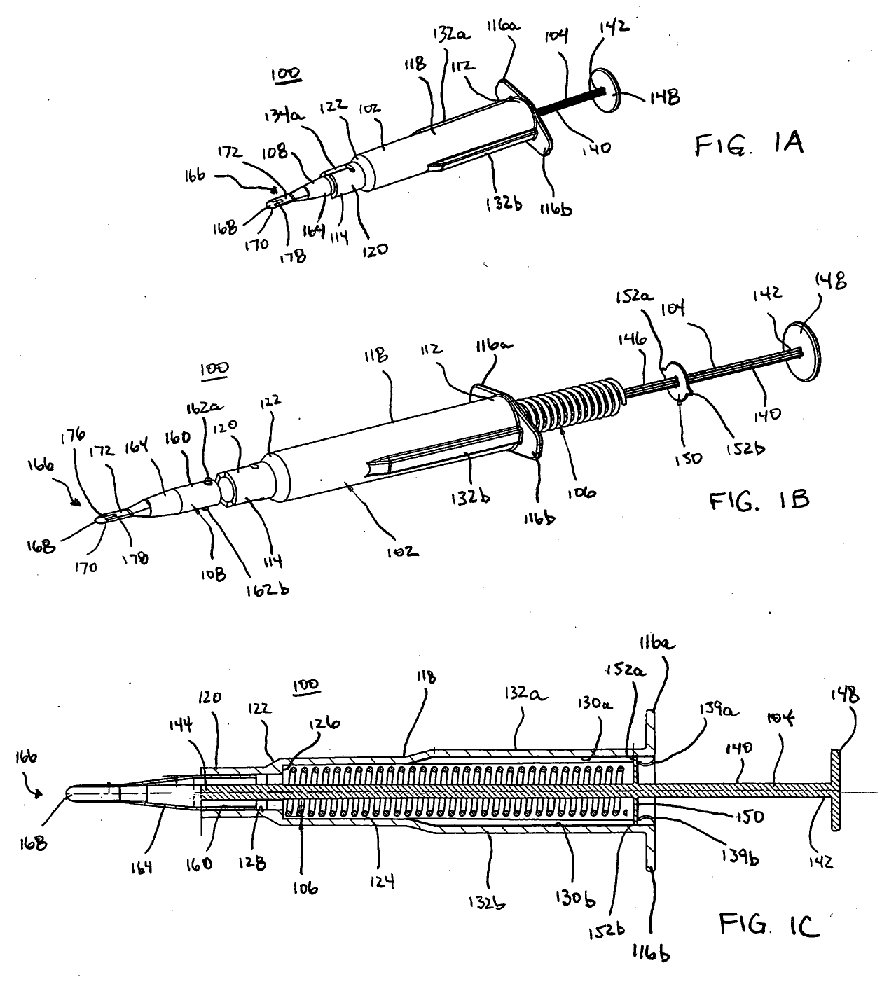

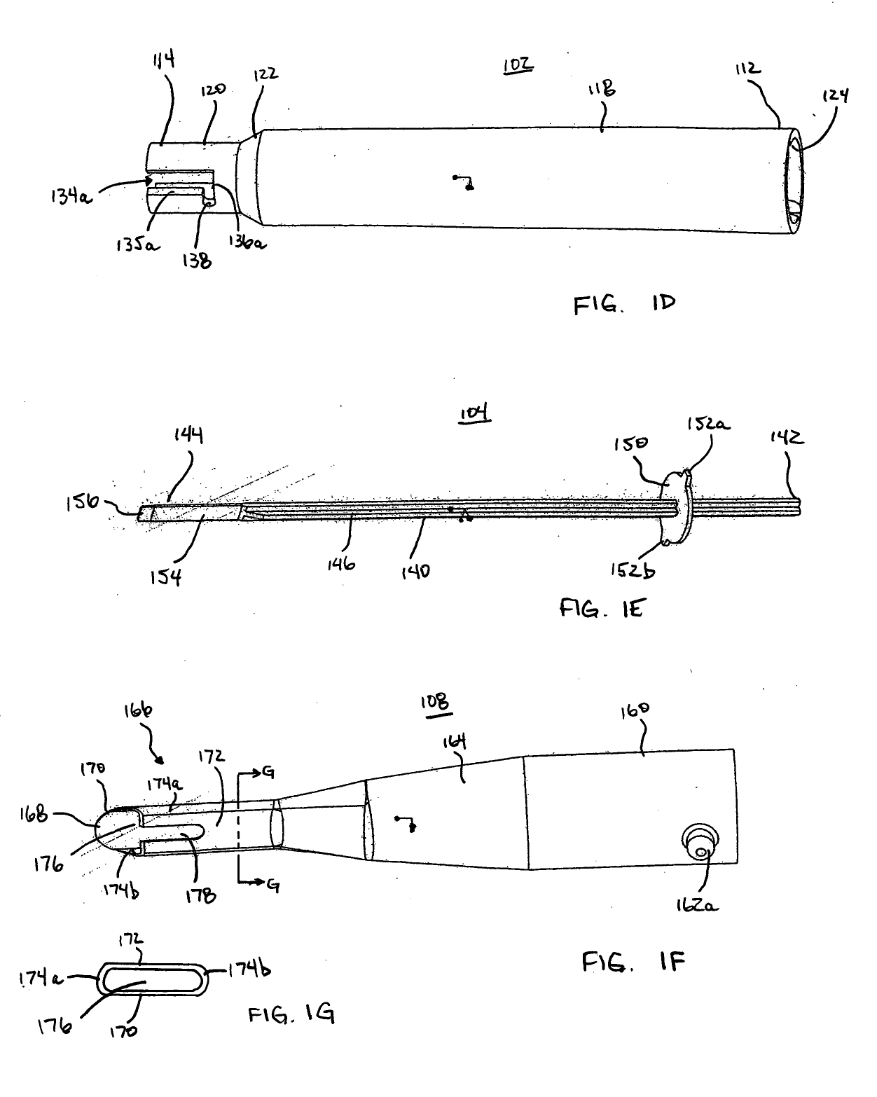

Turning now to the Figures, FIGS. 1A through 1C show an injector assembly that is particularly suited for depositing a corneal implant onto a corneal surface or beneath a flap or pocket formed on the surface of a cornea during a refractive surgical procedure. The injector assembly 100 includes an elongated barrel 102, a plunger 104 that extends longitudinally inside the barrel, a generally cylindrical spring 106 that also extends longitudinally inside the barrel coaxially with the plunger, and a detachable tip 108 attached to the distal end of the barrel.

The barrel 102 has a proximal end 112 and a distal end 114. A pair of finger grips 116a, 116b are formed integrally with the barrel 102 and project radially outward from near the proximal end 112 of the barrel. As shown, the finger grips 116a-b are generally flat and planar, lying in a plane that is generally perpendicular to the longitudinal axis of the barrel. Alternative shapes and orientations are also possible for the finger grips, such as curved surfaces or the like.

The barrel 102 includes a first portion 118 having a first inner and outer diameters, and a second portion 120 having a second inner and outer diameters, with the first inner and outer diameters being larger than the second inner and outer diameters. A transition 122 or step provides a transition between the first portion 118 and the second portion 120 of the barrel on the external surface of the barrel. As shown in FIG. 1C, the interior of the first portion 118 of the barrel provides a first cylindrical housing 124 that extends from the proximal end 112 of the barrel to a shoulder 126 formed near the transition 122 from the barrel first portion 118 to the barrel second portion 120. A second cylindrical housing 128, of a smaller diameter, is formed by the interior of the second portion 120 of the barrel and extends from the transition 122 to the distal end 114 of the barrel.

A pair of slots 130a-b are formed on opposed sides of the internal surface of the barrel first portion 118. The slots 130a-b extend from the proximal end 112 of the barrel for approximately half of the length of the barrel. As explained below, the length of the slots 130a-b will determine, in part, the amount of longitudinal travel for the plunger 104. A pair of fins 132a-b are formed on the external surface of the barrel first portion 118 to accommodate the slots 130a-b formed on the interior of the barrel.

One or more L-shaped slots 134a-b are formed at the distal end 114 of the barrel. (See FIG. 1D). Each slot includes a longitudinal section 135a-b extending longitudinally from the distal end of the barrel, and a transverse section 136a-b extending perpendicularly from the proximal end of each of the longitudinal sections 135a-b. A slightly rounded pocket 138a-b is formed at the end of each of the transverse sections 136a-b. As described more fully below, the L-shaped slots are adapted to provide an attachment mechanism for detachably attaching the injector tip 108 to the barrel 102.

The barrel 102 is preferably formed of a resilient plastic or other polymeric material, such as polypropylene (PP), polyvinyl acetate (PVA), polyvinyl chloride (PVC), polyethylene, polyester, nylon or polyamide, or any of a number of plastic materials known to those of ordinary skill in the art as suitable for use in various medical instruments and other similar devices. Alternatively, the barrel may be formed of a metal such as stainless steel or other metallic material.

The plunger 104 is an elongated member having a profile adapted to slide easily within both the first cylindrical housing 124 and second cylindrical housing 128 of the barrel 102. The plunger 104 includes an elongated shaft 140 having a proximal end 142 and a distal end 144. The cross-section of the shaft 140 may be round, square, rectangular, or other shape. As shown in FIGS. 1A, 1B, and 1E, the shaft includes a ridge 146 extending over a portion of its length from the proximal end, but terminating prior to the distal end. The ridge 146 may be used to provide additional structural strength, to provide an orientation mechanism for the plunger shaft, or to serve as a guide by cooperating with a mating slot (not shown) provided on the interior surface of the barrel. The distal end 147 of the ridge may be located so as to limit the amount of travel available to the plunger by engaging an interior surface of the barrel 102 or the detachable tip 108, as described more fully below.

A thumbpad 148 is formed at the proximal end 142 of the plunger shaft. The thumbpad 148 is a generally flat, planar member that extends generally perpendicularly from the longitudinal axis of the plunger shaft. Although the thumbpad 148 shown in FIGS. 1A-C is generally round in shape, other shapes or sizes may be used.

A spring block 150 is formed at approximately a mid-point on the length of the plunger shaft. The spring block 150 is a generally planar member that extends perpendicularly from the longitudinal axis of the shaft. The spring block 150 is generally round in shape to fit within the first cylindrical housing 124 of the barrel, and is provided with one or more tabs 152a-b on opposite sides of the spring block 150. The tabs 152a-b are adapted to slide within the pair of slots 130a-b formed on the internal surface of the barrel 102. As described below, the spring block 150 is adapted to engage the distal end of the spring 106 within the barrel 102.

The plunger includes a flat portion 154 extending over a portion of the length of the distal end 144 of the plunger shaft. The flat portion 154 is adapted to slide within the interior surface of the injector tip 108, as described more fully below. An implant engagement tip 156 is formed at the distal end of the flat portion 154, and is sized and shaped to provide a suitable surface for engaging and sliding a corneal implant from a storage location within the injector tip to its delivery location.

Like the barrel, the plunger 104 is preferably formed of a resilient plastic or other polymeric material, such as polypropylene (PP), polyvinyl acetate (PVA), polyvinyl chloride (PVC), polyethylene, polyester, nylon or polyamide, or any of a number of plastic materials known to those of ordinary skill in the art as suitable for use in various medical instruments and similar devices. Alternatively, the plunger 104 may be formed of a metal such as stainless steel or other metallic material.

The spring 106 is a generally cylindrical spring having a size adapted to fit readily within the cylindrical housing 124 formed on the interior of the barrel 102. When the injector assembly is fully assembled, the distal end of the spring 106 rests against the 126 formed within the cylindrical housing 124 of the barrel, and the proximal end of the spring 106 rests against the inner surface of the spring block 150 formed on the plunger 104. In this way, the spring 106 provides a force biasing the plunger 150 (and therefore the plunger 104) proximally.

As best seen in FIG. 1C, a pair of retainers 139a-b are formed on the interior surface of the barrel 102 near its proximal end. Each of the retainers extends inwardly from the interior surface of the barrel and engages the upper surface of the plunger spring block 150, thereby holding the spring block 150 (and plunger 104) in place against the proximal biasing force of the spring 106.

The spring 106 is preferably formed of a resilient plastic or other polymeric material, such as polypropylene (PP), polyvinyl acetate (PVA), polyvinyl chloride (PVC), polyethylene, polyester, nylon or polyamide, or any of a number of plastic materials known to those of ordinary skill in the art as suitable for use in various medical instruments and similar devices. Alternatively, the spring 106 may be formed of a metal such as stainless steel or other metallic material.

The injector tip 108 has a size and shape that allows it to detachably engage the distal end of the barrel 102. The tip 108 includes a first barrel portion 160 that has an outer diameter that is slightly smaller than the inner diameter of the second portion 120 of the barrel, to thereby provide a slidable fit between the two members. One or more knobs 162a-b are formed on opposed sides of the first barrel portion 160, with each knob having a shape and size adapted to engage the L-shaped slots 134a-b formed on the distal end of the barrel 102. To attach the injector tip 108 to the barrel 102, the knobs 162a-b are advanced longitudinally through the length of the longitudinal sections 135a-b, then the injector tip 108 is rotated to cause the knobs to slide through the transverse sections 136a-b until they engage the rounded pockets 138a-b. At this point, the injector tip 108 is engaged.

The external surface of the injector tip 108 is provided with a gradually narrowing transition region 164 and a distal region 166. The distal region 166 includes a flat tongue 168 having a rounded end. The distal region also includes a lower wall 170 that extends proximally from the tongue 168, an upper wall 172 that is spaced apart from and above the lower wall 170, and a pair of side walls 174a-b extending between and connecting together the lower wall 170 and upper wall 172. (See FIGS. 1F-G). The lower wall 170, upper wall 172, and side walls 174a-b together define a channel 176 within the injector tip 108. As can be best seen in FIG. 1G, the channel 176 has a width dimension “w” that is substantially larger than the height dimension “h”. For corneal implant injectors, typical sizes for the width dimension of the channel can range from about 2 mm to about 6 mm, and preferably from about 3 mm to about 5 mm. For these same corneal implant injectors, the typical sizes for the height dimension of the channel can range from about 200 microns to about 1.2 mm, and preferably about 600 microns to about 1 mm. The channel 176 provides a space within which an implant, such as a corneal implant, may be stored and from which the implant may be deployed. The upper wall 172 is provided with an optional cutout portion 178 that may serve as an access point or a viewing port to observe the contents of the channel 176.

Like the barrel, the plunger, and the spring, the injector tip 108 is preferably formed of a resilient plastic or other polymeric material, such as polypropylene (PP), polyvinyl acetate (PVA), polyvinyl chloride (PVC), polyethylene, polyester, nylon or polyamide, or any of a number of plastic materials known to those of ordinary skill in the art as suitable for use in various medical instruments and similar devices. Alternatively, the injector tip 108 may be formed of a metal such as stainless steel or other metallic material.

The injector assembly 100 is suitable both for storing an implant (such as a corneal implant) prior to its use, as well as for deployment (such as implantation) of the implant. For example, a corneal implant may be placed within the channel 176 of the detachable tip 108. Typically, the corneal implant will be placed in a compacted state, such as by curling the edge of the implant, prior to placement of the implant into the channel 176. See, for example, FIGS. 6A and 6B, where there is shown a corneal implant 190 in its expanded state (FIG. 6A), and in a compacted state (FIG. 6B). The compacted state has relatively smaller width w_{c} than the width of the corneal implant in its expanded state wₙ. This type of storage allows use of an injector having a relatively narrower deployment tip than would be needed if the corneal implant were to be stored in its expanded state. Advantageously, the corneal implant 190 will automatically expand to its full deployment state when it is released from the channel 176 during the deployment operation.

Additional information relating to corneal implants suitable for use with the injector assemblies described herein is provided in U.S. patent application Ser. No. ______, entitled “Implantable Lenses with Modified Edge Regions,” filed on Apr. 15, 2005 (Attorney Docket No. 15838.4021). The foregoing application is hereby incorporated by reference herein in its entirety.

It is preferable to store hydrophilic implants in a suitable liquid storage medium prior to deployment. The present injector assembly provides a ready method for doing so. For example, after an implant has been loaded into the channel 176 of the injector tip 108, the tip 108 may be detached from the barrel 102 (if it was attached) and placed in a vial or bottle of storage medium until it is needed. When it is needed, the injector tip 108 is attached to the barrel 102 of an injector assembly, and the assembly is ready for use.

As noted previously, the injector assembly is particularly suited for use in deploying a corneal implant during a refractive surgical procedure. In such procedures, a flap or pocket is formed on the cornea to provide access to a bed or channel onto or into which a corneal implant is to be deployed or implanted. Several methods for forming flaps in corneal tissue, and other related information, are described in further detail in co-pending U.S. patent application Ser. No. 10/924,152, filed Aug. 23, 2004, entitled “Method for Keratophakia Surgery,” which is fully incorporated by reference herein. In particular, the foregoing application describes methods for forming geometrically specific flaps in corneal tissue using a laser, such as a femtosecond laser. Alternatively, methods for forming a pocket, i.e., a recess formed in the corneal tissue without the formation of a “flap,” are described in United States Patent Application Publication No. 2003/0014042, published Jan. 16, 2003, entitled “Method of Creating Stromal Pockets for Corneal Implants,” which is also fully incorporated by reference herein. The foregoing published application describes methods for forming a stromal pocket using a pulsed laser beam. Once the stromal pocket is established, an entry channel extending from the anterior surface of the eye to the stromal pocket is created.

After formation of the flap or pocket, the injector assembly 100 is brought into position by placing the tongue 168 of the injector tip onto or near the deployment location on the corneal bed (in the case of a flap procedure) or in the corneal channel (in the case of a pocket procedure). The plunger 104 is then depressed by applying pressure on the thumbpad 148. Depression of the plunger 104 causes the implant engagement tip 156 to engage the corneal implant 190 and to eject the implant from the channel 176 onto the bed or into the channel formed on the cornea. The injector assembly 100 may then be withdrawn, after which the clinician may make any fine positional adjustments that may be necessary to place the implant in its proper orientation. The fine adjustments may be made with any conventional tool, such as a lint-free sponge or round-tipped tool.

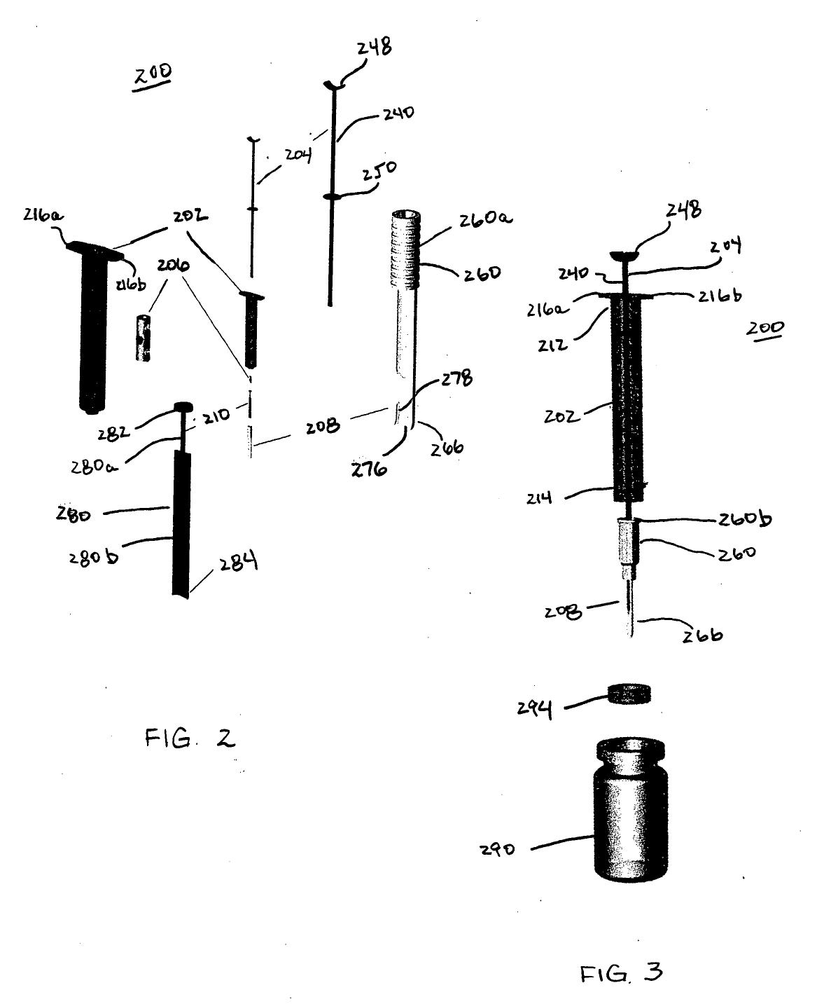

Another embodiment of an injector assembly is illustrated in FIGS. 2, 3, and 4A-C. In this embodiment, the injector assembly 200 includes a barrel 202, a handpiece plunger 204, a resilient spring 206, an insertion tip 208, and a tip plunger 210. The insertion tip 208 is detachable from the barrel 202 for ease of storage, as explained more fully below.

The barrel 202 is generally cylindrical, having a proximal end 212 and a distal end 214. A pair of finger grips 216a-b are formed on the proximal end 212 of the barrel, extending generally perpendicular to the longitudinal axis of the barrel 202. Both the internal and external surfaces of the barrel 202 are generally smooth and cylindrical. The barrel is preferably formed of a suitable plastic or metallic material, such as those described previously.

The handpiece plunger 204 includes an elongated shaft 240 having a generally round cross-sectional shape. The handpiece plunger 204 includes a curved thumbpad 248 formed at the proximal end of the shaft 240, and a disc-shaped spacer 250 formed at approximately a mid-point of the length of the plunger 204. The spacer 250 had a diameter that is slightly smaller than the internal diameter of the barrel 202, thereby providing a stabilizing force to the plunger 204 as it travels the length of the barrel 202. The plunger is preferably formed of a suitable plastic or metallic material, such as those described previously.

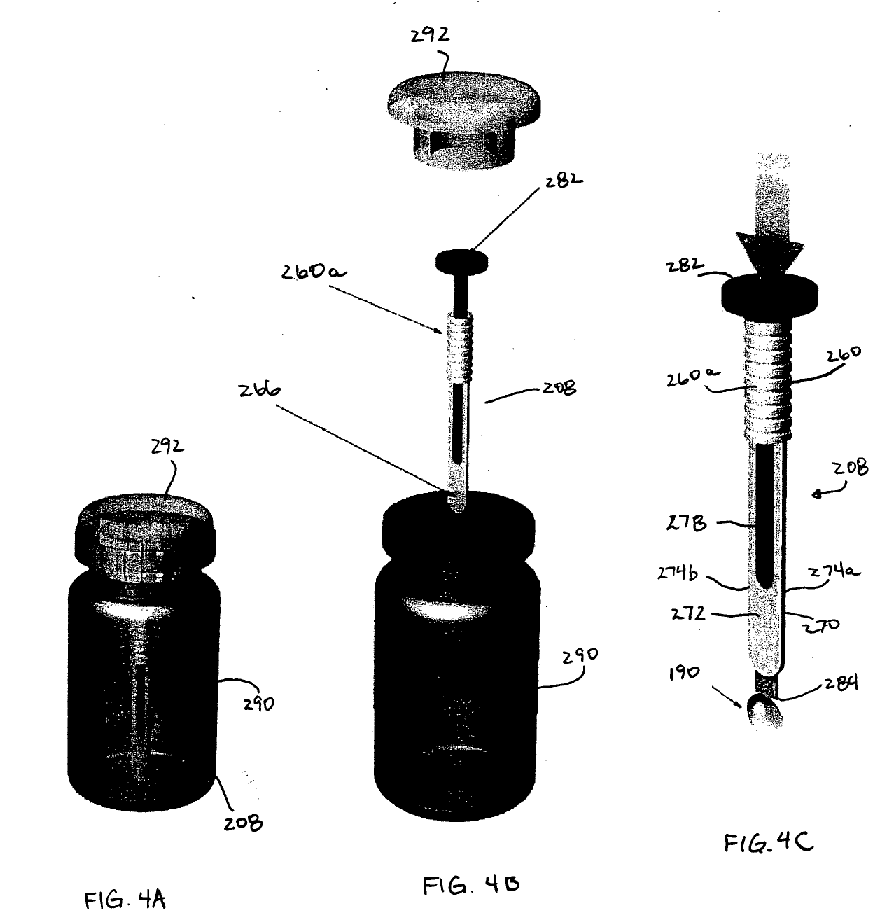

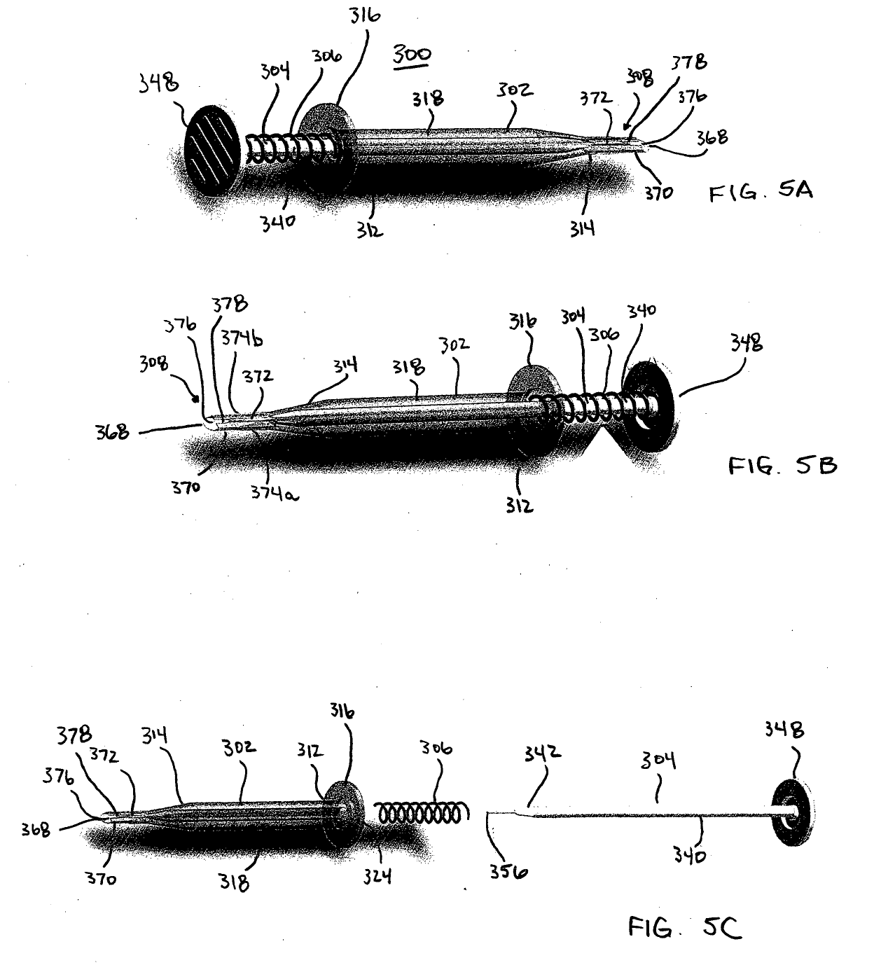

The insertion tip 208 includes a hub portion 260 for connecting to the distal end of the barrel 202, and a distal portion 266 for storing and delivering the implant, such as a corneal implant. The hub portion 260 may be provided with a plurality of ribs 260a, such as shown in FIGS. 2 and 4B-C, or it may include a luer fitting 260b, such as shown in FIG. 3. In either case, the internal surface of the distal end of the barrel 202 is provided with a mating feature to provide an adequate attachment between the barrel 202 and the insertion tip 208. Other mating fixtures will be recognize by persons of ordinary skill in the art as being suitable to provide the desired attachment mechanism.

The distal portion 266 of the insertion tip is constructed similarly to the distal region 166 of the injector assembly shown in FIGS. 1A-F described above. The distal portion includes a tongue 268 at the distal end of the insertion tip, a lower wall 270, upper wall 272, and a pair of side walls 274a-b which together define a channel 276 adapted to store and then deploy a suitable implant, such as a corneal implant. The upper wall 272 includes a cutout portion 278 to provide access and visibility to the interior of a portion of the channel 276.

The tip plunger 210 has a generally elongated shaft 280 that includes a first narrow region 280a near the proximal end of the tip plunger, and a second broader region 280b that extends distally from the narrow region. A pusher pad 282 is formed at the proximal end of the tip plunger 210. The pusher pad 282 is a flat, disc-shaped member that extends in a plane that lies perpendicular to the longitudinal axis of the tip plunger. The tip plunger includes an implant engagement tip 284 that has a size, shape, and orientation that is adapted to engage and safely push the implant through and out of the channel 276 formed in the insertion tip. For example, the implant engagement tip 284 may include a slit to form a soft, lint-free brush.

The resilient spring 206 is a generally cylindrical member formed of a highly elastic plastic, rubber, or other suitable material. Alternatively, the resilient spring 206 may be of a conventional coiled spring structure. The spring 206 is located concentrically with the narrow region 280a of the tip plunger just distally of the pusher pad 282, with the distal end of the spring 206 resting against a shoulder formed on the internal surface of the insertion tip hub 260 and the proximal end of the spring resting against the distal surface of the pusher pad 282. In this manner, the resilient spring 206 provides a force biasing the tip plunger proximally.

As noted previously, it is preferable to store corneal (and other) implants in a suitable liquid storage medium prior to deployment. The present injector assembly provides another ready method for doing so. For example, after an implant has been loaded into the channel 276 of the injector tip 208, the tip 208 (with the tip plunger 210 in place) may be detached from the barrel 202 (if it was attached) and placed in a vial or bottle of storage medium until it is needed. As shown, for example, in FIGS. 4A-B, a storage vial 290 may be charged with a suitable storage medium for storing the implant contained in the tip 208, and the tip 208 is then inserted into the vial 290 for storage. A stopper 292 is used to seal the vial and protect its contents. An optional stabilizer 294 may be placed at the mouth of the vial in order to hold the injector tip 208 in place within the vial 290. The stabilizer 294 may comprise a resilient cylindrical member having an opening adapted to engage the hub 260 of the injector tip.

When the injector assembly 200 is needed for use, the injector tip 208 may be connected to the handpiece portion, which includes the barrel 202 and the handpiece plunger 204, by attaching the injector tip hub 260 to its mating fitting on the distal end of the barrel 202. (See FIG. 3). The injector assembly 200 is then ready for use.

As noted previously, the injector assembly 200 is particularly suited for use in deploying a corneal implant during a refractive surgical procedure. In such procedures, a flap or pocket is formed on the cornea to provide access to a bed or channel onto or into which a corneal implant is to be deployed or implanted, as described above. After formation of the flap or pocket, the injector assembly 200 is brought into position by placing the tongue 268 of the injector tip onto or near the deployment location on the corneal bed (in the case of a flap procedure) or in the corneal channel (in the case of a pocket procedure). The handpiece plunger 204 is then depressed by applying pressure on the thumbpad 248. Depression of the handpiece plunger 204 causes the distal end of the handpiece plunger 204 to engage the pusher pad 282 of the tip plunger 210 and to force the tip plunger 210 distally against the biasing force of the resilient spring 206. As the tip plunger 210 moves distally, the implant engagement tip 256 engages the corneal implant 190 and ejects the implant from the channel 276 onto the bed or into the channel formed on the cornea. The injector assembly 200 may then be withdrawn, after which the clinician may make any fine positional adjustments that may be necessary to place the implant in its proper orientation. The fine adjustments may be made with any conventional tool, such as a lint-free sponge or round-tipped tool.

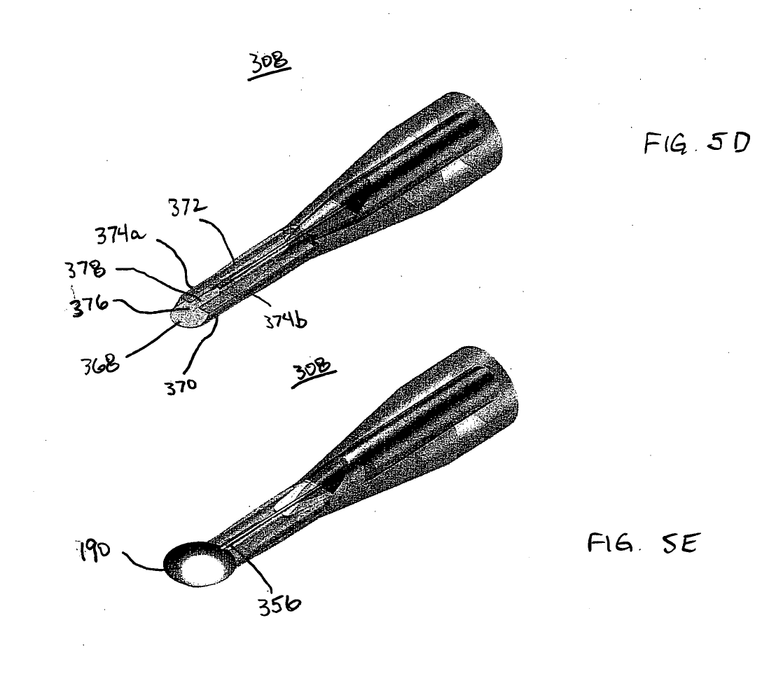

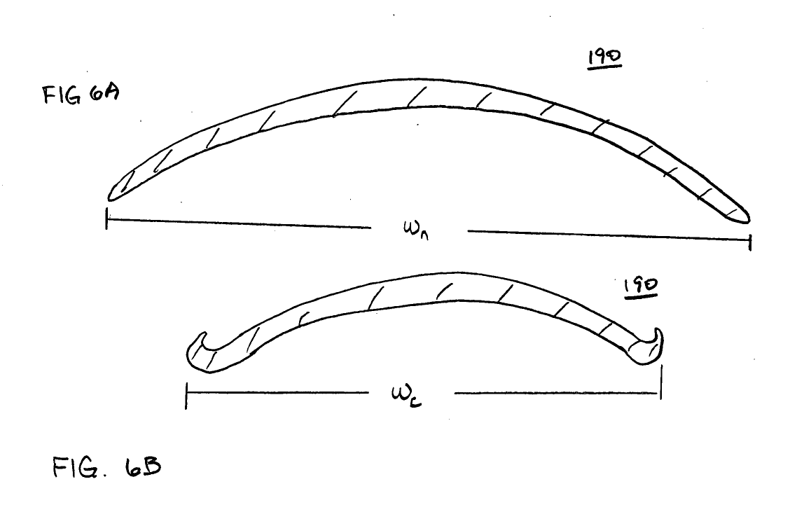

Another embodiment of an injector assembly is illustrated in FIGS. 5A-E. In this embodiment, the injector assembly 300 includes a barrel 302, a plunger 304, and a spring 306. An injector tip region 308 is formed integrally with the barrel 302 at the distal end 314 of the barrel. Thus, in this embodiment, the injector tip is not detachable from the handpiece portion of the assembly.

The barrel 302 is generally cylindrical, having a proximal end 312 and a distal end 314. A first portion 318 of the barrel has generally smooth and cylindrical internal and external surfaces having a first inner and outer diameters. The distal region 314 of the barrel has a gradual taper on its external surface that interconnects the barrel first portion 318 with the tip region 308. A finger grip 316 is formed on the proximal end 312 of the barrel, extending generally perpendicular to the longitudinal axis of the barrel 302. The finger grip 316 is a generally planar, disc-shaped member that extends over the entire periphery of the proximal end 312 of the barrel. The barrel is preferably formed of a suitable plastic or metallic material, such as those described previously.

The plunger 304 includes an elongated shaft 340 having a generally round cross-sectional shape over most of its length. The plunger 304 includes a generally disc-shaped thumbpad 348 formed at the proximal end of the shaft 340. The distal end of the plunger is provided with an implant engagement tip 356, which has a shape, size, and orientation adapted to safely engage and move a corneal implant within the channel 376 provided on the injector tip region 308. For example, the implant engagement tip 356 may include a soft sponge. The plunger shaft 340 also includes a tapered region 342 connecting the main portion of the shaft with the implant engagement tip. The plunger is preferably formed of a suitable plastic or metallic material, such as those described previously.

The tip region 308 of the barrel is constructed similarly to the distal region 166 of the injector assembly shown in FIGS. 1A-F described above. The distal portion includes a tongue 368 at the distal end of the injector assembly, a lower wall 370, upper wall 372, and a pair of side walls 374a-b which together define a channel 376 adapted to store and then deploy a suitable implant, such as a corneal implant. The upper wall 372 includes a cutout portion 378 to provide access and visibility to the interior of a portion of the channel 376.

The spring 306 is a generally cylindrical spring having a size adapted to fit readily within the cylindrical housing 324 formed on the interior of the barrel 302. When the injector assembly is fully assembled, the distal end of the spring 306 rests against a shoulder 326 formed within the cylindrical housing 324 of the barrel, and the proximal end of the spring 306 rests against the distal surface of the thumbpad 348 formed on the plunger 304. In this way, the spring 306 provides a force biasing the plunger 304 proximally.

As noted previously, it is preferable to store hydrophilic implants (including corneal implants) in a suitable liquid storage medium prior to deployment. The present injector assembly provides another ready method for doing so. For example, after an implant has been loaded into the channel 376 of the injector tip 308, the entire injector assembly may be placed in a vial or bottle of storage medium until it is needed. Under such storage conditions, it is expected that the liquid storage medium may enter the interior of the barrel 302 and cover all of the surfaces of the injector assembly. This causes no undesirable effects and does not detract from the performance of the apparatus. When the injector assembly 300 is needed for use, the assembly is simply removed from the storage container and it is ready for use.

As noted previously, the injector assembly 300 is particularly suited for use in deploying a corneal implant during a refractive surgical procedure. In such procedures, a flap or pocket is formed on the cornea to provide access to a bed or channel onto or into which a corneal implant is to be deployed or implanted, as described above. After formation of the flap or pocket, the injector assembly 300 is brought into position by placing the tongue 368 of the injector tip onto or near the deployment location on the corneal bed (in the case of a flap procedure) or in the corneal channel (in the case of a pocket procedure). The plunger 304 is then depressed by applying pressure on the thumbpad 348. Depression of the plunger 304 causes the implant engagement tip 356 to engage the corneal implant 190 and ejects the implant from the channel 376 onto the bed or into the channel formed on the cornea. (See FIG. 5E). The injector assembly 300 may then be withdrawn, after which the clinician may make any fine positional adjustments that may be necessary to place the implant in its proper orientation. The fine adjustments may be made with any conventional tool, such as a lint-free sponge or round-tipped tool.

Several modifications and additions to the above embodiments are possible. For example, in the embodiment described above in reference to FIGS. 5A-E, the spring 306 may be replaced by a bellows structure 390 interconnecting the plunger thumbpad 348 and the proximal end 312 of the barrel. See FIG. 7. The bellows structure 390 includes an integrated bellows formed of a resilient, elastic material, such as silicone, rubber, or other polymeric material. The bellows structure 390 provides a force biasing the plunger 304 proximally, thereby providing a resistive force during deployment of the implant.

An additional modification is illustrated in FIG. 8, where there is shown an injector assembly that includes an implant adjustment member 400 incorporated into the apparatus. The adjustment member 400 includes a flat, elongated tongue 402 that is able to be extended and retracted from the distal end of the injector assembly. The tongue 402 may also be curved, or may have another shape and orientation suitable to provide the implant adjustment function. The tongue 402 is attached to a lever 404 that extends through a longitudinal slot 406 formed in the barrel 302. Longitudinal movement of the lever 404 causes the tongue 402 to extend or retract from the distal end of the injector assembly. Preferably, the tongue 402 is located in a plane just above the upper wall 372 of the tip region 308 of the assembly.

The implant adjustment member 400 assists in protecting and delivering the implant during insertion onto or into the corneal tissue. The moveable tongue 402 protects the implant during insertion by keeping the implant sandwiched between the implant adjustment member tongue 402 and the injector assembly tongue 368, then enables the implant to be delivered into place by moving the moveable tongue 402 away from the implant, thereby placing the implant into the soft tissue and allowing it to adhere to the tissue, while the injector assembly is removed from the tissue.

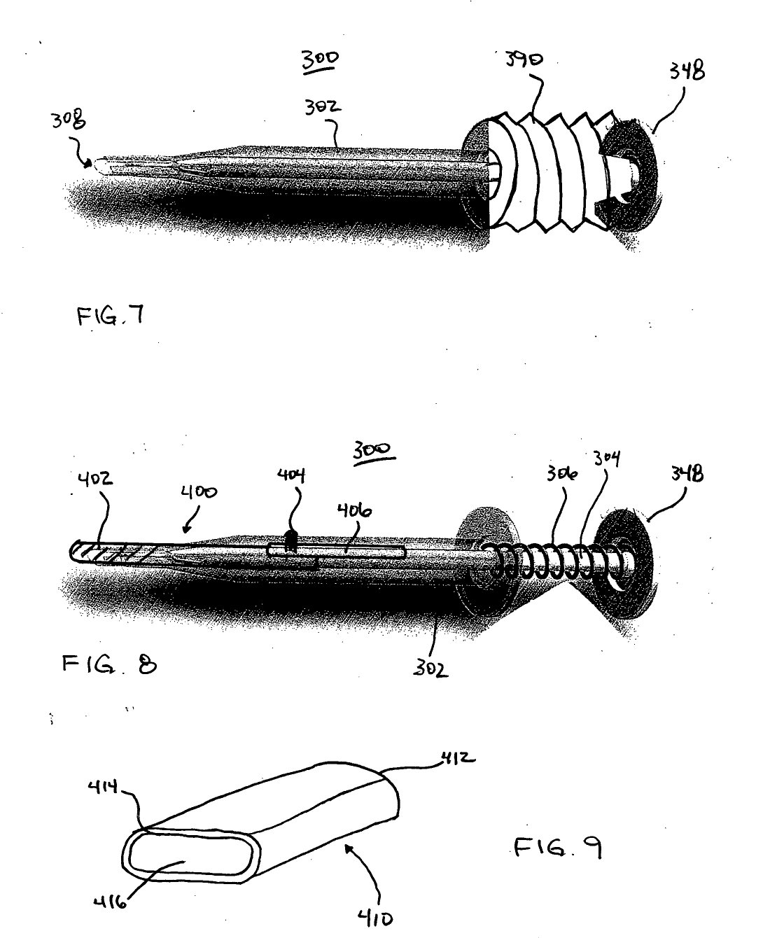

A cap may be attached to the tip portion of any of the injector assembly embodiments described above. A representative cap is illustrated in FIG. 9. The cap 410 is preferably an elongated member having a closed distal end 412 and an open proximal end 414 that provides access to an internal space 416 defining a protective housing. The exterior of the cap 410 may be generally cylindrical, conical, or other shape. The cap is adapted to slide over the tip portion of the injector assembly and to remain in place until the injector assembly is ready to be used. The internal space 416 preferably has dimensions and is oriented to provide a friction fit engagement with the distal region of the injector tip of each of the injector assembly embodiments described above. Alternatively, the cap may be provided with a positive attachment mechanism, such as a tab that engages a mating slot on the injector assembly, or other similar mechanism known to those skilled in the art. The cap may serve to protect the injector tip from damage during shipping and prior to use, and also may assist in retaining the implant 190 within the injector tip during storage prior to use. The cap preferably is made from a plastic material such as those described above, or other material suitable for use in the manner described.

The preferred embodiments of the inventions that are the subject of this application are described above in detail for the purpose of setting forth a complete disclosure and for the sake of explanation and clarity. Those skilled in the art will envision other modifications within the scope and spirit of the present disclosure. Such alternatives, additions, modifications, and improvements may be made without departing from the scope of the present inventions, which is defined by the claims.

What is claimed is:

1. A corneal implant delivery system, comprising:
a plunger contained within a housing and capable of being advanced distally within the housing, the plunger having a distal end;
a tip portion associated with a distal end of the housing, said tip portion having an upper wall and lower wall defining a channel, and a tongue portion extending distally of the channel from one of said upper wall and said lower wall; and
a corneal implant retained within the tip portion channel, said corneal implant being in a contracted state while retained within the tip portion channel;
wherein the distal end of said plunger engages said corneal implant when the plunger is advanced distally.

2. The implant delivery system of claim 1, wherein said tongue portion comprises a flat member having a rounded distal edge.

3. The implant delivery system of claim 1, further comprising first and second side walls connecting the upper wall and lower wall of the tip portion, said first and second side walls being generally parallel to one another, said upper wall and said lower wall being generally parallel to one another, and said first and second sidewall and said upper wall and said lower wall defining the channel.

4. The implant delivery system of claim 3, wherein a first distance between said upper wall and said lower wall of the tip portion is smaller than a second distance between said first side wall and said second side wall of the tip portion.

5. The implant delivery system of claim 4, wherein said first distance is between about 2.0 mm and about 6.0 mm, and said second distance is between about 200 microns and about 1.2 mm.

6. The implant delivery of system of claim 1, wherein said tip portion is formed integrally with the housing.

7. The implant delivery system of claim 1, wherein said tip portion is detachable from the housing.

8. The implant delivery system of claim 7, wherein said plunger comprises a first plunger portion contained within said housing, and a second plunger portion contained within said tip portion.

9. The implant delivery system of claim 1, further comprising a spring member interposed between a portion of said housing and a portion of said plunger, said spring member providing a force biasing said plunger proximally relative to said housing.

10. The implant delivery system of claim 1, further comprising an elongated implant positioning member associated with said housing, said implant positioning member being capable of movement relative to said tip portion.

11. The implant delivery system of claim 10, wherein said implant positioning member further comprises a lever extending through a slot on said housing.

12. A method of placing a corneal implant onto a corneal tissue surface comprising:
forming a site on or in a cornea for receiving a corneal implant;
positioning a corneal implant delivery system proximal to the site, said corneal implant system comprising a tip portion containing a corneal implant and a plunger; and
actuating the plunger of said delivery system to cause the corneal implant to be discharged from the tip portion of the delivery system onto or into the site.

13. The method of claim 12, further comprising positioning the corneal implant on or in the site.

14. The method of claim 12, wherein said forming step comprises performing a surgical procedure upon the cornea.

15. The method of claim 14, wherein said surgical procedure comprises forming a corneal flap.

16. The method of claim 14, wherein said surgical procedure comprises forming a corneal pocket.
